(19) United States
(12) Patent Application Publication
(10) Pub. No.: US 2009/0324516 A1
(43) Pub. Date: Dec. 31, 2009

(54) Composition and Method for Enhancing Flouride Uptake Using Bioactive Glass

(76) Inventors: Dave P. MUSCLE, Alachua, FL (US); Anora K. Burwell, San Francisco, CA (US); Gaetano LaTorre, Gainesville, FL (US)

Correspondence Address: BRINKS, HOFER, GILSON & LIONE, P.O. BOX 1340, MORRISVILLE, NC, 27560, US

(21) Appl. No.: 12/492,302

(22) Filed: Jun. 26, 2009

Related U.S. Application Data
(60) Provisional application No. 61/076,210, filed on Jun. 27, 2008.

(51) Int. Cl. A61K 8/21; A61Q 11/00

(52) U.S. Cl. 424/52

(57) ABSTRACT

A method for increasing fluoride uptake onto a tooth structure of a patient comprises contacting the tooth structure with an oral care composition, which comprises bioactive glass and fluoride. The oral care composition has the following formulation:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

### RELATED APPLICATIONS

The present patent document claims the benefit of the filing date under 35 U.S.C. §119(e) of Provisional U.S. Patent Application Ser. No. 61/076,210, filed Jun. 27, 2008, which is hereby incorporated by reference.

### FIELD OF THE INVENTION

The invention relates to enhancing fluoride uptake into teeth. Specifically, the invention relates to a composition for and a method of enhancing fluoride uptake into a tooth using bioactive glass.

### BACKGROUND

Dentin and enamel in teeth are composed primarily of crystalline calcium phosphate in the form of hydroxyapatite. At normal oral pH levels, this tooth mineral is highly insoluble. However, at acidic pH levels, significant demineralization and mineral loss can occur. Saliva, which is naturally supersaturated with calcium and phosphorous ions, is normally responsible for remineralizing and repairing tooth surfaces. As a result of poor oral care habits and modern diets, teeth are constantly demineralized by cariogenic bacteria and acidic food and drink leaving the normal repair process out of balance. In addition, diseases, such as neck and throat cancers, diabetes, and high blood pressure, and the medications that are taken to combat certain illnesses also contribute to the break down of the normal repair process. One method used to reduce the amount of demineralization of the tooth surface is to introduce fluoride into the oral environment in the form of fluoride toothpaste and rinses. The fluoride from these products is ionized by saliva and incorporated into the tooth structure in the form of hydroxyfluoroapatite. Hydroxyfluoroapatite is much more acid resistant than hydroxyapatite thereby reducing the rate of demineralization of the tooth surface caused by further acid challenges.

It is known that conventional fluoride compounds, in the form of sodium fluoride, sodium monofluorophosphate, stannous fluoride, and other fluoride compounds, can be incorporated into oral care compositions to enhance the remineralization and acid resistance of tooth minerals. It is also known that abrasives, in the form of calcium carbonate, dicalcium phosphate, tricalcium phosphate, and other calcium sources can release low levels of calcium and enhance the uptake of fluoride into tooth mineral. Using poorly soluble calcium sources like the abrasives mentioned above, results in low levels of calcium being released into the saliva thereby resulting in marginal improvements in the fluoride uptake into the tooth structure. The use of highly soluble calcium containing compounds, in the form of calcium chloride, calcium citrate, amorphous calcium phosphate (ACP), casein phosphopeptide amorphous calcium phosphate (CPP-ACP), and other highly soluble calcium compounds has been proposed to enhance fluoride uptake into the tooth mineral. Directly combining fluoride containing compounds with highly soluble calcium containing compounds in oral care compositions can be difficult because the soluble fluoride and calcium can react in the package to form insoluble calcium fluoride, thus reducing the efficacy of the fluoride when it enters the oral environment. These highly soluble compounds can also react when introduced into the oral environment resulting in the formation of calcium fluoride and reducing the fluoride uptake into the tooth mineral. A number of approaches to developing viable oral care compositions including these two sources have been attempted.

One approach is to use oral care compositions in series, i.e., one followed by the other, rather than simultaneously. This approach allows for the separate introduction of soluble calcium, phosphorous, and fluoride into the saliva which then can react to enhance fluoride uptake into the tooth structure. For example, U.S. Pat. Nos. 4,083,955 (Grabenstetter et al) and 4,397,837 (Raaf et al), describe a process for remineralizing enamel by the consecutive treatment of tooth surfaces with separate solutions containing calcium ions and phosphate ions. This method of treatment has the inconvenience of a plurality of sequential applications which are time consuming and inconvenient.

A second approach is to develop oral care compositions with low pH to enhance the solubility of fluoride, calcium, and phosphorous compounds into the saliva to enhance fluoride uptake into the tooth structure. U.S. Pat. No. 4,080,440 (Dugiulio et al) discloses a metastable solution of calcium and phosphate ions at low pH (between 2.5 to 4.0). Penetration of the solution into demineralized enamel occurs, and remineralization occurs from the precipitation of calcium phosphate when pH rises. Unfortunately, metastable solutions lower pH, which can potentially demineralize dentin and enamel and/or injure or irritate soft oral tissues, if incorporated into daily use products like toothpaste.

Yet another approach is to use a dual phase delivery system that keeps the soluble fluoride and calcium separated until the point of use. U.S. Pat. No. 4,397,837 (Raaf et al), U.S. Pat. No. 6,485,708 (Winston et al.), and U.S. Pat. No. 5,891,448 (Chow et al.) disclose dual phase delivery systems. Dual delivery systems can be problematic with regard to accurate, proper dosage and delivery of suitable fluoride compounds. Further, additional costs are associated with dual delivery systems because of the extra materials and packaging required for dual delivery systems.

U.S. Pat. Nos. 5,437,857; 5,460,803; 5,871,360; 6,000,341; and 6,056,930 attributed to Tung and the American Dental Association involve new compositions and methods of use and delivery of amorphous calcium compounds such as amorphous calcium phosphate (ACP), amorphous calcium phosphate fluoride (ACPF), amorphous calcium carbonate phosphate (ACCP), amorphous calcium carbonate phosphate fluoride (ACCPF), and amorphous calcium fluoride (ACF) for use in remineralizing and fluoridating teeth. The compounds claim the highest solubilities, fastest formation rates, and fastest conversion rates of all remineralizing products. However, high solubility is potentially disadvantageous because it prevents prolonged deposition of the disclosed calcium compounds onto the tooth surface, thus preventing the compounds from aiding in remineralizing and fluoridating the tooth mineral. In addition, these highly soluble compounds are also problematic because the soluble calcium that is released from the compound can inactivate the fluoride in a formulation by forming insoluble calcium fluoride.

Thus, there is a need to develop oral care compositions including soluble sources of fluoride, calcium, and phosphorous that is highly efficacious and overcomes the drawbacks of currently available products and solutions.

### SUMMARY OF THE INVENTION

In an aspect of the invention, a method for increasing fluoride uptake onto a tooth structure of a patient comprises contacting the tooth structure with an oral care composition comprising bioactive glass and fluoride. In a feature of the invention the fluoride is in the bioactive glass. In another feature of the invention, the oral care composition comprises the following formulation:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

In a further feature, the enamel fluoride concentration of the tooth structure after being contacted with the oral care composition is over 900 ppm. In still a further feature, the oral care composition comprises the following formulation:

| Ingredient | Weight Percent |
|---|---|
| Glycerin | 45-65 |
| PEG 400 | 15-25 |
| Silica - Abrasive | 5-20 |
| Silica - Thickening | 3-8 |
| Bioactive glass | 3-10 |
| Surfactant | 0-2 |
| Colorant | 0-2 |
| Flavor | 0.1-2 |
| Sodium Monofluorophosphate | 0-1 |
| Gum Binder | 0-1 |
| Sweetener | 0-1 |

In additional features, the oral care composition is a dentifrice and a dental varnish. In a further feature, the surfactant is selected from the group consisting of polyethoxylated sorbitol monoesters, Tween, polycondensates of ethylene oxide and propylene oxide (poloxamers), condensates of propylene glycol, polyethoxylated hydrogenated castor oil, and sodium lauryl sulphate. In another feature, the gum binder is selected from the group consisting of carboxyvinyl polymers, carrageenans, hydroxyethylcellulose, carboxymethylcellulose (CMC), karaya, xanthan, gum arabic, and tragacanth. In yet another feature, the sweetener is selected from the group consisting of saccharin, cyclamate, potassium acesulfame, xylitol, and thaumatin.

In a second aspect of the invention, an oral care composition comprises the following formulation:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

In a feature of this aspect, the oral care composition comprises the following formulation:

| Ingredient | Weight Percent |
|---|---|
| Glycerin | 45-65 |
| PEG 400 | 15-25 |
| Silica - Abrasive | 5-20 |
| Silica - Thickening | 3-8 |
| Bioactive glass | 3-10 |
| Surfactant | 0-2 |
| Colorant | 0-2 |
| Flavor | 0.1-2 |
| Sodium Monofluorophosphate | 0-1 |
| Gum Binder | 0-1 |
| Sweetener | 0-1 |

In additional features, the oral care composition is a dentifrice, a dental varnish, and a sealant. In a further feature, the surfactant is selected from the group consisting of polyethoxylated sorbitol monoesters, Tween, polycondensates of ethylene oxide and propylene oxide (poloxamers), condensates of propylene glycol, polyethoxylated hydrogenated castor oil, and sodium lauryl sulphate. In another feature, the gum binder is selected from the group consisting of carboxyvinyl polymers, carrageenans, hydroxyethylcellulose, carboxymethylcellulose (CMC), karaya, xanthan, gum arabic, and tragacanth. In yet another feature, the sweetener is selected from the group consisting of saccharin, cyclamate, potassium acesulfame, xylitol, and thaumatin.

In a third aspect of the invention, a dentifrice comprises the following formulation:

| Ingredient | Weight Percent |
|---|---|
| Glycerin, 99.7% | 55.38 |
| PEG 400 | 20.00 |
| Silica - Abrasive | 10.00 |
| Silica - Thickening | 5.00 |
| Bioactive glass | 5.00 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.50 |
| Potassium Acesulfame | 0.40 |

In a fourth aspect of the invention, a dental varnish comprises the following formulation:

| Ingredient | Weight Percent |
|---|---|
| Colophony Resin | 25-75 |
| Ethyl Alcohol | 5-25 |
| Bioactive glass | 5-20 |
| Silica - Thickening | 0.5-5 |
| Sodium Fluoride | 0-5 |
| Flavor | 0.5-2.0 |
| Sweetener | 0.5-5 |

### DETAILED DESCRIPTION OF THE INVENTION

The present invention relates to a non-aqueous oral care composition comprising bioactive glass and fluoride. As will be explained in greater detail below, additional components may be included in the oral care composition. In addition, it will be understood by one of ordinary skill in the art that an oral care composition may include dentifrices, dental varnishes, dental sealants, chewing gums, dissolvable strips, mouthwashes, and other fluoride containing oral care products. The oral care composition should be formulated and manufactured in a way that prevents the bioactive glass from reacting with the formulation thereby releasing calcium and phosphorous in the formulation and reacting with the fluoride source.

An exemplary formulation for the oral care composition is as follows:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

It will be understood by one of ordinary skill in the art that components that may be included for some embodiments of the oral care composition may not be included for other embodiment of the oral care composition. For example, a dentifrice may comprise glycerin, PEG, an abrasive, a thickening agent, a surfactant, and other components, and a varnish may comprise colophony resin and ethyl alcohol, while not comprising PEG or glycerin. However, all oral care compositions according to the invention will include bioactive glass and fluoride.

Fluoride uptake onto tooth surfaces is affected by calcium and phosphate concentrations in saliva. Bioactive glass releases calcium, phosphorous, sodium, and silicon ions into aqueous solutions. Thus, when bioactive glass is included in a fluoride oral care composition, for example, a dentifrice, the release of supplemental calcium and phosphorus from the bioactive glass advantageously increases the uptake of fluoride onto tooth surfaces. The release of these ions can also elicit a modest pH rise that has the potential to increase remineralization in the oral environment.

As used herein, the term “increasing fluoride uptake onto a tooth structure” means increasing or enhancing the amount of fluoride that is absorbed or bound onto the tooth surface or tooth structure. The fluoride concentration in the tooth enamel can be measured to determine the amount of fluoride uptake onto a tooth structure. FDA Monograph 40 provides a method for measuring enamel fluoride concentration.

As used herein, the terms “tooth structure” and “tooth surface” mean any part of an individual's teeth to which fluoride may be absorbed or bound. As such, tooth structure and tooth surface includes, but is not limited to, tooth enamel, incipient enamel lesions, hydroxyapatite in the enamel, dentin, and cementum.

As used herein, the term “non-aqueous” means anhydrous or substantially free of water. The individual components of the non-aqueous composition may contain limited amounts of water as long as the overall composition remains substantially free of water.

As used herein, the term “oral care composition” includes any preparation used in all or a portion of the oral cavity of an individual for improving or maintaining overall good general health in the oral cavity. For example, an oral care composition may enable or aid in improving or maintaining good oral hygiene, preventing or reducing decay, preventing or reducing gingivitis and/or plaque, remineralizing the tooth surface, and treating dentin hypersensitivity.

As used herein, the term “dentifrice” includes any preparation used in cleansing all or a portion of the oral cavity of an individual.

As used herein, the term “dental varnish” includes a composition topically applied to the tooth surface for fluoride therapy. Typically, a dental varnish includes a high concentration of fluoride.

As used herein, the term “oral cavity” means an individual's teeth, and gums, including all periodontal regions including teeth down to the gingival margins and/or the periodontal pockets.

As used herein the term “bioactive glass” means an inorganic glass material having an oxide of silicon as its major component and which is capable of bonding with growing tissue when reacted with physiological fluids. By way of example, a bioactive glass in accordance with the invention is a glass composition that will form a layer of hydroxyapatite in vitro when placed in a simulated body fluid. A bioactive glass as used herein is also biocompatible such that it does not trigger an overwhelmingly adverse immune response in the body, such as in the oral cavity.

Bioactive glasses are well known to those skilled in the art, and are disclosed, for example, in An Introduction to Bioceramics, L. Hench and J. Wilson, eds. World Scientific, New Jersey (1993), the entire contents of which is hereby incorporated by reference.

The following composition, shown in weight percent of each element in oxide form, provides a bioactive glass:

| | |
|---|---|
| SiO₂ | 40-86 |
| CaO | 4-35 |
| Na₂O | 0-35 |
| P₂O₅ | 2-15 |
| CaF₂ | 0-25 |
| B₂O₃ | 0-10 |
| K₂O | 0-8 |
| MgO | 0-5 |

The same bioactive glass composition can be expressed in weight percent of each element as follows:

| | |
|---|---|
| Si | 19-40 |
| Ca | 1-6 |
| Na | 0-26 |
| P | 1-7 |
| Ca | 0-13 |
| B | 0-3 |
| K | 0-8 |
| Mg | 0-3 |
| F | 0-12 |
| O | Balance |

The bioactive glass can be manufactured using a “melting process” or a “sol-gel” process, both of which are well known to those skilled in the art of making bioactive glass.

Bioactive glasses are considered a class A bioactive material that will bond to both hard and soft tissue. As such bioactive glasses provide a more efficacious material for interaction with the tooth structure. Bioactive glass may be present in the oral care composition formulation in an amount from about 0.5 to 15 weight percent of the oral care composition, preferably from about 3 to 10 weight percent of the oral care composition.

The oral care composition formulation further comprises a fluoride-containing compound, which may include ionic fluorides, such as alkali metal fluorides, amine fluorides and ionic monofluorophosphates, such as alkali metal monofluorophosphates, and which may be incorporated into the formulation, to provide between about 100 and 5000 ppm, preferably about 500 to 2000 ppm of fluoride. The fluoride compound may comprise sodium fluoride or sodium monofluorophosphate. Stannous fluoride may also be used at the above levels. Calcium glycerophosphate, which has been shown to enhance the activity of ionic monofluorophosphates, may be optionally added when the fluoride source is an ionic monofluorophosphate. Fluoride may be present as a separate fluoride source in the oral care composition formulation in an amount from about 0 to 5.0 weight percent of the oral care composition.

As an alternative to the use of a separate fluoride source in the oral care composition, the use of a fluoride containing bioactive glass, such as the bioactive glass in U.S. Pat. No. 4,775,646, which is incorporated by reference herein, may also be used as a fluoride source where the fluoride from the bioactive glass is released into the oral environment and the bioactive glass composition enhances the fluoride uptake into the tooth surface.

It will further be appreciated that if an ionic fluorine-containing compound is incorporated in an oral care composition of the invention, an abrasive should be chosen so that it is compatible with the ionic fluorine-containing compound. Thus, for instance, sodium fluoride is well known in the art to be incompatible with abrasives that comprise excess calcium ions as these cause loss of fluoride as insoluble calcium fluoride. Accordingly, an abrasive which is insoluble, for instance, a silica, alumina, zinc orthophosphate or plastic particles, is preferred. Alternatively, a calcium abrasive, for instance calcium carbonate or di-calcium phosphate, may be used with an alkali metal monofluorophosphate, e.g., sodium monofluorophosphate.

The oral care composition may further comprise a carboxyvinyl polymer. The carboxyvinyl polymer is used in the acid form and does not necessarily require any form of neutralizing. Carboxyvinyl polymers thicken humectant materials and also provide the preferred rheology in order to suspend any required abrasive material. The term ‘rheology’ as used herein is intended to reflect the flow characteristics of the formulation.

Suitable carboxyvinyl polymers for use in the oral care compositions are copolymers of acrylic acid cross-linked with polyallylsucrose, for example, Carbopol 974 and 934 or cross-linked with divinyl glycol, for example, Noveon AA-1. Carbopol polymers are manufactured by B.F. Goodrich Company. Carbopol 974 is preferred. Carboxyvinyl polymer may be present in a range of from 0.1 to 7.5 weight percent of the oral care composition, preferably from 0.3 to 1.0 weight percent, more preferably about 0.5 weight percent of the oral care composition.

Other natural and synthetic polymers may be used alone or in combination in the oral care compositions. Exemplary polymers include, but are not limited to, carrageenans, hydroxyethylcellulose, carboxymethylcellulose (CMC) and natural gum binders including karaya, xanthan, gum arabic, and tragacanth.

The oral care composition may further comprise a humectant. Suitable humectants for use in the oral care composition include glycerine, sorbitol, and propylene glycol or mixtures thereof. It is well known that commercially available glycerine may contain between 0.5-2.0 weight percent of water in association with the glycerine. Typically this amount is between 0.5-1.0 weight percent. This small amount of water is bound to the glycerine and is therefore unavailable to the other ingredients. As such, the skilled person would consider a composition containing glycerine as being nonaqueous. The humectants should be as anhydrous as possible and preferably used in solid form. Glycerine is a preferred humectant. As the humectant is used to make the formulations up to 100%, the humectant may be present in ranges from about 20 to 90 weight percent of the oral care composition. Preferably, the humectant is present from about 35 to 75 weight percent, more preferably from about 45 to 75 weight percent of the oral care composition.

The oral care composition may further comprise polyethylene glycol. The polyethylene glycol is selected so that it will reduce any stickiness from the formulation and give a smooth textured product. The polyethylene glycol will be selected from a molecular weight range of PEG 300 to PEG 1000. PEG 400 is preferred. Advantageously, the polyethylene glycol is present in ranges from about 0.1 to 40 weight percent, preferably about 15 to 20 weight percent of the oral care composition.

The oral care composition may further comprise an abrasive. Suitable abrasives for use in the oral care composition include, for example, silica, zinc orthophosphate, sodium bicarbonate (baking soda), plastic particles, alumina, hydrated alumina, calcium carbonate and calcium pyrophosphate or mixtures thereof. The silica abrasive may be a natural amorphous silica, for instance diatomaceous earth; a synthetic amorphous silica, such as a precipitated silica, for instance ‘Tixosil 53B’, manufactured by Rhone Poulenc; a silica gel, such as a silica xerogel; or mixtures thereof. Generally, an amount of abrasive suitable for use in the dentifrice composition of the present invention will be empirically determined to provide an acceptable level of cleaning and polishing, in accordance with the techniques well known in the art. Suitably, the abrasive will be present in from about 0 to about 60 weight percent, preferably from about 0 to about 30 weight percent of the oral care composition. Notwithstanding the above, it will be understood by one of ordinary skill in the art that additional abrasives, such as those listed above, may not be needed for the oral care composition because bioactive glass has an intrinsic abrasive characteristic.

Advantageously, a thickening agent may be present in the oral care composition of the invention to give the oral care composition a rheology closer to that of a conventional oral care composition. The thickening agent can be a thickening silica, for instance ‘Sident 22S’, which is manufactured by Degussa Ltd. The thickening silica can be in the range of from about 0.01 to 10 weight percent, preferably about 5.0 to 7.0 weight percent of the oral care composition.

The oral care composition may further comprise a surfactant. Surfactant materials are usually added to oral care composition products to provide cleaning and/or foaming properties. Any conventional surfactant used in oral care composition formulations may be used in the present invention, provided that it can be added as a solid powder that is not in an aqueous solution. Suitable surfactants include anionic, cationic, nonionic and amphoteric surfactants.

Suitable nonionic surfactants include, for example, polyethoxylated sorbitol esters, in particular polyethoxylated sorbitol monoesters, for instance, PEG(40) sorbitan diisostearate, and the products marketed under the trade name ‘Tween’ by ICI; polycondensates of ethylene oxide and propylene oxide (poloxamers), for instance the products marketed under the trade name ‘Pluronic’ by BASF-Wyandotte; condensates of propylene glycol; polyethoxylated hydrogenated castor oil, for instance, cremophors; and sorbitan fatty esters. Suitable anionic surfactants include, for example sodium lauryl sulphate, marketed by Albright and Wilson and known as ‘SLS’. This may be obtained and is used in a powder form in the present invention. Advantageously, the surfactant is present in the range of about 0 to 20 weight percent, preferably about 0 to 10 weight percent, more preferably about 0 to 2.5 weight percent of the oral care composition.

The oral care composition may also comprise other agents conventionally used in oral care composition formulations at appropriate levels, for example, coloring agents, including whitening agents, such as titanium dioxide, hydrogen peroxide, and sodium tripolyphosphate; preservatives; and sweetening agents. Anti-plaque agents, for example, triclosan, chlorhexidine, cetyl pyridinium chloride and nicin (preferably in a purified form, and available as Ambicin N); anti-calculus agents, for example, pyrophosphate salts; anti-sensitivity agents, for example strontium or potassium salts; polymer enhancing agents, for example Gantrez may also be present if required. Breath freshening agents, for example, sodium bicarbonate, may also be included at appropriate levels. In general, such agents will be in a minor amount or in proportion to the formulation, usually present in from about 0.001 to 5 weight percent of the composition. Any active ingredient or combination of actives that are unstable or incompatible in any way with aqueous environments may also be added to the formulation of the present invention. That is to say, any active ingredient or combination of active ingredients that are stable or compatible in any way with non-aqueous environments may also be added to the formulation of the present invention.

Flavoring agents may also be added to the oral care composition formulations, usually at a concentration of about 1.0 weight percent of the oral care composition. It will be understood that flavoring agents may also be added at different concentrations. Suitable sweetening agents may include saccharin, cyclamate, and potassium acesulfame and may be present in from about 0.01 to 1.0 weight percent, preferably 0.05 to 0.5 weight percent of the oral care composition. Alternatively, xylitol, which is not an intense sweetener, can be used as a sweetener in concentrations from about 1.0 to about 15 weight percent of the oral care composition. An auxiliary sweetener such as a thaumatin may also be included, at a level of from 0.001 to 0.1, preferably 0.005 to 0.05 weight percent of the oral care composition. A suitable blend of thaumatins is marketed under the trade name ‘TALIN’ by Tate and Lyle PLC.

The oral care composition may further comprise an antistain agent. Suitable antistain agents include, for example, carboxylic acids such as those disclosed in U.S. Pat. No. 4,256,731, amino carboxylate compounds such as those disclosed in U.S. Pat. No. 4,080,441 and phosphonoacetic acid, as disclosed in U.S. Pat. No. 4,118,474. The antistain agent may be incorporated into the oral care composition formulation or may be provided as a separate composition, for use after the oral care composition.

The oral care composition, particularly a dental varnish, may comprise a resin, which can serve as a film former. A preferred resin is colophony resin. Colophony is a natural resin derived from living trees. Colophony resin may be present in from about 25 to 75 weight percent of the oral care composition. Although a colophony resin is preferred, resorbable and biocompatible polymers can also be used.

The oral care composition, particularly a dental varnish, may comprise an alcohol, which can serve as a solvent. A preferred alcohol is ethyl alcohol. Ethyl alcohol may be present in from about 5 to 25 weight percent of the oral care composition.

The oral care composition may have an initial viscosity of about 25,000 to 400,000 centipoise, which is comparable to the viscosity of conventional oral care compositions that have consumer acceptability. The pH of the formulation, when diluted in the ratio of about 3:1 with water, should be less than 10.0. The viscosity of the oral care composition is measured using a TF 20 spindle Brookfield Viscometer.

The oral care composition may be prepared in a conventional manner by mixing the ingredients thereof in the appropriate proportions and in any order that is convenient and, thereafter, if necessary, adjusting the pH. In a particularly preferred process for preparing a dentifrice, the polyvinyl polymer and the humectant are vigorously agitated together, with heat, for example to a temperature of, for example 50° to 70° C., if necessary, in order to give a satisfactory viscosity. Polyethylene glycol and thickening silica are then added to the mixture and abrasive is then dispersed in it, using a heavy-duty mixing machine. Active agents, such as a fluoride salt (if present) are then added, followed by surfactant and flavoring agents in the final stage. Final mixing is carried out under vacuum.

### EXAMPLES

Enamel fluoride uptake is an in vitro method that was designed to evaluate how much fluoride is absorbed on the tooth surface from fluoride-containing dentifrices. Standard in vitro methods have also been developed to evaluate the total fluorine content and total soluble available fluoride in fluoride-containing dentifrices.

The following examples illustrate the invention.

### Example 1

An in vitro study was performed with five different dentifrice formulations to determine the effect of the dentifrices on promoting fluoride uptake into incipient enamel lesions. The test procedure for determining enamel fluoride uptake was identical to the one identified as Procedure 40 in the FDA Monograph except the lesion was formed using a solution that was 0.1M lactic acid and 0.2% Carbopol 907 and was 50% saturated with HAP at a pH of 5.0. Total fluorine was tested using FDA Monograph method 3, and total soluble available fluoride was tested using FDA Monograph method 16.

Sound, upper, central, bovine incisors were selected and cleaned of all adhering soft tissue. A core of enamel 3 mm in diameter was prepared from each tooth by cutting perpendicular to the labial surface with a hollow-core diamond drill bit. This was performed under water to prevent overheating of the specimens. Each specimen was embedded in the end of a plexiglass rod (¼″ diameter×2″ long) using methylmethacrylate. The excess acrylic was cut away exposing the enamel surface. The enamel specimens were polished with 600 grit wet/dry paper and then with micro-fine Gamma Alumina. The resulting specimen was a 3 mm disk of enamel with all but the exposed surface covered with acrylic.

Each enamel specimen was then etched by immersion into 0.5 ml of 1M HCIO4 for 15 seconds. Throughout the etching period, the etch solutions were continuously agitated. A sample of each solution was then buffered with TISAB to a pH of 5.2 (0.25 ml sample, 0.5 ml TISAB, and 0.25 ml 1N NaOH) and the fluoride content determined by comparison to a similarly prepared standard curve (1 ml std and 1 ml TISAB). For use in depth of etch calculation, the Ca content of the etch solution was determined by taking 50 μl and analyzing for Ca by atomic absorption (0.05 ml qs to 5 ml). These data were the indigenous fluoride level of each specimen prior to treatment.

The specimens were once again ground and polished as described above. An incipient lesion was formed in each enamel specimen by immersion into a 0.1M lactic acid/0.2% Carbopol 907 solution for 24 hours at room temperature. These specimens were then rinsed well with distilled water and stored in a humid environment until used.

The treatments were performed using supernatants of the dentifrice slurries. The slurries consisted of 1 part dentifrice and 3 parts (9 g:27 ml, w/w) distilled water. Each slurry was mixed well for 30 seconds and then centrifuged for 10 minutes at about 10,000 rpm. The rapid mix time was because, once extruded, the test product contains available Ca and is meant to be activated in the mouth during use. If the traditional longer mix times were used without the enamel specimens being in contact with the slurry, the test product would have been at an unfair disadvantage as compared to the control. The specimens were then immersed into 25 ml of their assigned supernatant with constant stirring (350 rpm) for 30 minutes. Following treatment, the specimens were rinsed with distilled water. One layer of enamel was then removed from each specimen and analyzed for fluoride and calcium, as outlined above (i.e., 15 second etch). The pretreatment fluoride (indigenous) level of each specimen was then subtracted from the post treatment value to determine the change in enamel fluoride due to the test treatment. In order to determine the change in enamel fluoride, enamel fluoride uptake (EFU), total fluorine (TF; 1:100 dilution), and total soluble available fluoride (TSAF; 1:10 dilution), were measured using FDA methods #40, #3, and #16, respectively. Results were analyzed using ANOVA and Newman-Keuls methods (p<0.01).

The dentifrices used for testing were as follows: 1) RD07344—placebo; 2) USP Reference Dentifrice (#1277401 1000 SMFP/Silica, Lot PTG 07-04); 3) RD07338—5% bioactive glass, 1000 ppm fluoride, 15% silica base formula; 4) RD07339—7.5% bioactive glass, 1000 ppm fluoride, 15% silica base formula; and 5) RD07341—5% bioactive glass, 1000 ppm fluoride, 18.5% silica base formula. Table 1 shows the composition for test dentifrice 1, RD07344—placebo. Table 2 shows the composition for test dentifrice 3, RD07338. Table 3 shows the composition for test dentifrice 4, RD07339. Table 4 shows the composition for test dentifrice 5, RD07341.

The testing results showed that the placebo dentifrice provided an EFU result (increase in enamel fluoride concentration) of 17±3 ppm. The USP Reference Dentifrice provided an EFU result of 686±15 ppm. Test dentifrice 3, RD07338, provided an EFU result of 929±26 ppm. Test dentifrice 4, RD07339, provided an EFU result of 901±29 ppm, and test dentifrice 5, RD07341, provided a fluoride uptake of 991±25 ppm. Test dentifrices 3-5 showed a marked increase in enamel fluoride uptake over the USP Reference Dentifrice. More particularly, test dentifrice 3 provided a greater than 35% increase in EFU, test dentifrice 4 provided a greater than 30% increase in EFU, and test dentifrice 5 provided a greater than 44% increase in EFU in comparison to the USP Reference Dentifrice. TF (Total Fluoride) results were: USP reference—1055±4 ppm F, dentifrice 3—936±7 ppm F, and dentifrice 4—935±9 ppm F. TSAF results were: USP reference—1023±7 ppm F, dentifrice 3—927±2 ppm F, and dentifrice 4—914±8 ppm F. These dentifrices met the FDA requirement for TF (850-1150 ppm F) and TSAF (Total Soluble Available Fluoride) (≧800 ppm F) in fresh silica-based SMFP dentifrices. More complete results of the testing are provided in Table 5.

TABLE 1
RD 07344 Composition

| Ingredient | Weight Percent |
|---|---|
| Glycerin, 99.7% | 56.15 |
| PEG 400 | 20.00 |
| Silica - Abrasive | 15.00 |
| Silica - Thickening | 5.00 |
| Sodium Lauryl Sulfate | 1.10 |
| Titanium Dioxide | 1.00 |
| Flavor | 0.85 |
| Carbopol 974P | 0.50 |
| Potassium Acesulfame | 0.40 |
| Total | 100.00 |

TABLE 2
RD 07338 Composition

| Ingredient | Weight Percent |
|---|---|
| Glycerin, 99.7% | 55.38 |
| PEG 400 | 20.00 |
| Silica - Abrasive | 10.00 |
| Silica - Thickening | 5.00 |
| Bioactive glass | 5.00 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.50 |
| Potassium Acesulfame | 0.40 |
| Total: | 100.00 |

TABLE 3
RD 07339 Composition

| Ingredient | Weight Percent |
|---|---|
| Glycerin, 99.7% | 52.88 |
| PEG 400 | 20.00 |
| Silica - Abrasive | 10.00 |
| Silica - Thickening | 5.00 |
| Bioactive glass | 7.50 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.50 |
| Potassium Acesulfame | 0.40 |
| Total: | 100.00 |

TABLE 4
RD 07341 Composition

| Ingredient | Weight Percent |
|---|---|
| Glycerin, 99.7% | 53.98 |
| PEG 400 | 18.00 |
| Silica - Abrasive | 15.00 |
| Bioactive glass | 5.00 |
| Silica - Thickening | 3.50 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.40 |
| Potassium Acesulfame | 0.40 |
| Total: | 100.00 |

TABLE 5
Fluoride uptake as affected by dentifrice composition.

| | Enamel Fluoride Concentration (ppm) | | |
|---|---|---|---|
| Dentrifice | Pre Ttreatment | Post Treatment | Increase |
| RD07344 | 39 ± 2 | 56 ± 3 | 17 ± 3 |
| placebo | | | |
| USP Reference | 38 ± 2 | 724 ± 16 | 686 ± 15 |
| Dentrifice | | | |
| (#1277401) | | | |
| RD07338 | 38 ± 2 | 967 ± 26 | 929 ± 26 |
| RD07339 | 41 ± 3 | 942 ± 28 | 901 ± 29 |
| RD07341 | 36 ± 2 | 1027 ± 26 | 991 ± 25 |

### Example 2

An in vitro study was performed with two fluoride varnish formulations to determine the effect of the addition of NovaMin® bioactive glass on fluoride ion release.

Two sample varnishes were tested: 1) a 0.100±0.002 grams of 10% NovaMin®±5% sodium fluoride varnish and 2) 5% sodium fluoride varnish. The varnish samples were applied to epoxy substrates and placed in 20 mL deionized (DI) water. The samples were placed in an incubator shaker at 37° C. and 200 rpm. The DI water was removed and replaced after 1, 4, 24, and 48 hours. The removed DI water was analyzed for ion content at each time interval.

To analyze ion content, the DI water samples were diluted 1:1 with a total ionic strength adjustment buffer (TISAB) and analyzed for fluoride ion concentration using a fluoride-selective electrode. Calcium and phosphorus ion concentrations were measured using inductively coupled plasma (ICP) spectroscopy. All results are presented as cumulative mean μg [ion]/g varnish (n=9). Additionally, the results were statistically analyzed using ANOVA and Student-Newman-Keuls methods (p<0.05).

The results, which are also shown in Table 6, were as follows: fluoride ion release from NovaMin®+fluoride varnish sample at 1, 4, 24, 48 hours: 97.8±1.5, 1,134.9±50.3, 9,835.8±52.3, 10,346.8±27.2; fluoride-only varnish sample: 71.4±7.4, 130.4±12.5, 301.7±41.1, 513.1±123.1. Calcium ion release from NovaMin®+fluoride sample: 31.3±4.0, 378.1±85.7, 1,166.7±81.4, 2,521.9±110.5; fluoride-only sample: 13.3±2.6, 52.1±2.3, 71.9±7.6, 65.4±6.9. Phosphorus ion release from NovaMin®+fluoride sample: 115.8±1.4, 340.4±8.8, 1,491.5±31.5, 1,618.2±34.2; fluoride-only sample: 112.5±0.5, 219.5±2.2, 307.6±2.5, 396.1±3.2. The NovaMin®-containing fluoride varnish had significantly higher release of all measured ions at all time points (p<0.05) compared to the fluoride-only varnish.

These results indicate that NovaMin®-containing fluoride varnishes exhibit increased fluoride release, which is likely to result in increased fluoride uptake and has the potential to remineralize tooth surfaces better than fluoride-only varnishes.

TABLE 6
Ion release as affected by NovaMin ® + fluoride varnish

| | NM + F | F-only | NM + F | F-only | NM + F | F-only |
|---|---|---|---|---|---|---|
| Time | F⁻ Release | F⁻ Release | Ca²⁺ Release | Ca²⁺ Release | P³⁺ Release | P³⁺ Release |
| (hrs) | (μg/g) | (μg/g) | (μg/g) | (μg/g) | (μg/g) | (μg/g) |
| 1 | 97.8 | 71.4 | 31.3 | 13.3 | 115.8 | 112.5 |
| 4 | 1,134.9 | 130.4 | 378.1 | 52.1 | 340.4 | 219.5 |
| 24 | 9,835.8 | 301.7 | 1,166.7 | 71.9 | 1,491.5 | 307.6 |
| 48 | 10,346.8 | 513.1 | 2,521.9 | 65.4 | 1,618.2 | 396.1 |

As shown, when tested using FDA monograph methods, adding bioactive glass to fluoride dentifrices significantly enhanced fluoride uptake into artificial carious lesions in enamel surfaces. Bioactive glass-containing fluoride dentifrices also met all FDA requirements for fluoride availability and release. These results indicate that bioactive glass-containing fluoride dentifrices may have a greater potential to fluorinate tooth surfaces than conventional fluoride-only dentifrices. A synergistic relationship between bioactive glass and fluoride is demonstrated in which bioactive glass provides the supplemental calcium and phosphorus needed for fluoride uptake into tooth surfaces therefore increasing potential for remineralization.

While the examples show enhanced fluoride uptake results for a dentifrice comprising bioactive glass and fluoride and increased ion release for a varnish comprising bioactive glass and fluoride, one of ordinary skill in the art would expect the same or similar fluoride uptake results and/or ion release results for any oral care composition comprising bioactive glass and fluoride, including, but not limited to, dental varnishes, dental sealants, chewing gums, dissolvable strips, mouthwashes, and other fluoride containing oral care products.

An exemplary formulation for a dentifrice is as follows:

| Ingredient | Weight Percent |
|---|---|
| Glycerin, 99.7% | 55.38 |
| PEG 400 | 20.00 |
| Silica - Abrasive | 10.00 |
| Silica - Thickening | 5.00 |
| Bioactive glass | 5.00 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.50 |
| Potassium Acesulfame | 0.40 |
| Total: | 100.00 |

An exemplary formulation for a dental varnish is as follows:

| Ingredient | Weight Percent |
|---|---|
| Colophony Resin | 25-75 |
| Ethyl Alcohol | 5-25 |
| Bioactive glass | 5-20 |
| Silica - Thickening | 0.5-5 |
| Sodium Fluoride | 0-5 |
| Flavor | 0.5-2.0 |
| Sweetener | 0.5-5 |

Topically-applied dental varnishes typically comprise a preparation of sodium fluoride in a resin carrier. Dental varnishes have been widely used to reduce tooth sensitivity and prevent caries for pediatric and high-risk caries patients. The resin carrier used in many commercial varnish products is a colophony resin, which is usually derived from the sap of living pine trees. For oral care applications, an esterification process is often used to modify the physical properties of the colophony resin, e.g., to lighten the color of the resin so that it more closely matches the color of the tooth surface for cosmetic reasons. Colophony resins can also be modified to increase their hydrophilic properties and to enhance adherence to the moist surface of the tooth by reacting it with maleic acid, maleic anhydride, or fumaric acid. Colophony resins may be present in dental varnishes in concentrations from about 20-75 weight percent of the varnish. Solvents, such as ethyl alcohol, may be used in concentrations of about 5-20 weight percent of the varnish to reduce the viscosity of the varnish for ease of application. The solvent evaporates after application, resulting in film formation of the varnish on the tooth surface. Fillers, such as silica, can be used in concentrations of around 0.5 to 5 weight percent of the varnish to enhance the viscosity and handling properties of the dental varnish. The varnish composition can also include coloring agents and flavors to enhance the appearance and taste of the product. A variety of fluoride sources can be used in a dental varnish formulation in concentrations of about 0.5 to 5 weight percent of the varnish.

Given the foregoing, using bioactive glasses in oral care compositions to enhance fluoride uptake will enable the development of low fluoride-containing oral care compositions. For example, in the United States, fluoride is used in concentrations from 900 ppm to 1450 ppm in consumer dentifrice formulations pursuant to regulatory requirements because these concentrations are the most efficacious. However, the use of fluoride at these high concentrations is of concern for some members of the population, for example, young children and hospitalized patients, due to concerns of fluoride ingestion and toxicity. Thus, the use of fluoride at concentrations below 900 ppm, but having the same efficacy as higher fluoride concentrations, is desirable.

It will be understood to those of ordinary skill in the art that the same can be performed within a wide and equivalent range of conditions and other parameters, as well as performed within a wide range of oral care product forms, without affecting the scope of the invention or any embodiment thereof. All patents, patent applications, and publications cited herein are fully incorporated by reference herein in their entirety.

What is claimed is:

1. A method for increasing fluoride uptake onto a tooth structure of a patient, comprising contacting the tooth structure with an oral care composition comprising bioactive glass and fluoride.

2. The method of claim 1, wherein the fluoride is in the bioactive glass.

3. The method of claim 1, wherein the oral care composition comprises the following formulation:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

4. The method of claim 1, wherein the enamel fluoride concentration of the tooth structure after being contacted with the oral care composition is over 900 ppm.

5. The method of claim 1, wherein the oral care composition comprises the following formulation:

| Ingredient | Weight Percent |
|---|---|
| Glycerin | 45-65 |
| PEG 400 | 15-25 |
| Silica - Abrasive | 5-20 |
| Silica - Thickening | 3-8 |
| Bioactive glass | 3-10 |
| Surfactant | 0-2 |
| Colorant | 0-2 |
| Flavor | 0.1-2 |
| Sodium Monofluorophosphate | 0-1 |
| Gum Binder | 0-1 |
| Sweetener | 0-1 |

6. The method of claim 5, wherein the surfactant is selected from the group consisting of polyethoxylated sorbitol monoesters, Tween, polycondensates of ethylene oxide and propylene oxide (poloxamers), condensates of propylene glycol, polyethoxylated hydrogenated castor oil, and sodium lauryl sulphate.

7. The method of claim 5, wherein the gum binder is selected from the group consisting of carboxyvinyl polymers, carrageenans, hydroxyethylcellulose, carboxymethylcellulose (CMC), karaya, xanthan, gum arabic, and tragacanth.

8. The method of claim 5, wherein the sweetener is selected from the group consisting of saccharin, cyclamate, potassium acesulfame, xylitol, and thaumatin.

9. The method of claim 5, wherein the oral care composition is a dentifrice.

10. The method of claim 5, wherein the oral care composition is a dental varnish.

11. An oral care composition comprising the following formulation:

| Ingredient | Weight Percent |
|---|---|
| Glycerin | 0-90 |
| PEG | 0-40 |
| Abrasive | 0-30 |
| Thickening Agent | 0.1-10 |
| Bioactive glass | 0.5-15 |
| Surfactant | 0-10 |
| Colorant | 0-2.0 |
| Flavor | 0.1-2.0 |
| Fluoride | 0-5.0 |
| Gum Binder | 0-2.0 |
| Sweetener | 0-1.0 |
| Colophony Resin | 0-75 |
| Ethyl Alcohol | 0-25 |

12. The oral care composition of claim 11, comprising the following formulation:

| Ingredient | Weight Percent |
|---|---|
| Glycerin | 45-65 |
| PEG 400 | 15-25 |
| Silica - Abrasive | 5-20 |
| Silica - Thickening | 3-8 |
| Bioactive glass | 3-10 |
| Surfactant | 0-2 |
| Colorant | 0-2 |
| Flavor | 0.1-2 |
| Sodium Monofluorophosphate | 0-1 |
| Gum Binder | 0-1 |
| Sweetener | 0-1 |

13. The composition of claim 12, wherein the surfactant is selected from the group consisting of polyethoxylated sorbitol monoesters, Tween, polycondensates of ethylene oxide and propylene oxide (poloxamers), condensates of propylene glycol, polyethoxylated hydrogenated castor oil, and sodium lauryl sulphate.

14. The composition of claim 12, wherein the gum binder is selected from the group consisting of carboxyvinyl polymers, carrageenans, hydroxyethylcellulose, carboxymethylcellulose (CMC), karaya, xanthan, gum arabic, and tragacanth.

15. The composition of claim 12, wherein the sweetener is selected from the group consisting of saccharin, cyclamate, potassium acesulfame, xylitol, and thaumatin.

16. The composition of claim 12, wherein the composition is a dentifrice.

17. The composition of claim 12, wherein the composition is a dental varnish.

18. The composition of claim 12, wherein the composition is a sealant.

19. A dentifrice comprising the following formulation:

| Ingredient | Weight Percent |
|---|---|
| Glycerin, 99.7% | 55.38 |
| PEG 400 | 20.00 |
| Silica - Abrasive | 10.00 |
| Silica - Thickening | 5.00 |
| Bioactive glass | 5.00 |
| Sodium Lauryl Sulfate | 1.10 |
| Titanium Dioxide | 1.00 |
| Flavor | 0.85 |
| Sodium Monofluorophosphate | 0.77 |
| Carbopol 974P | 0.50 |
| Potassium Acesulfame | 0.40 |

20. A dental varnish comprising the following formulation:

| Ingredient | Weight Percent |
|---|---|
| Colophony Resin | 25-75 |
| Ethyl Alcohol | 5-25 |
| Bioactive glass | 5-20 |
| Silica - Thickening | 0.5-5 |
| Sodium Fluoride | 0-5 |
| Flavor | 0.5-2.0 |
| Sweetener | 0.5-5 |
